(19) United States
(12) Patent Application Publication
(10) Pub. No.: US 2002/0081572 A1
(43) Pub. Date: Jun. 27, 2002

(54) Three dimensional structure of paramyxovirus hemagglutinin neuraminidases and use thereof

(76) Inventors: Garry Taylor, St. Andrews (GB); Allen Portner, Bartlett, TN (US); Toru Takimoto, Bartlett, TN (US); Y. Sidhakar Babu, Hoover, AL (US); R. Scott Rowland, Hoover, AL (US)

Correspondence Address: Burton A. Amernick, Connolly Bove Lodge & Hutz LLP, P.O. Box 19088, Washington, DC, 20036-0088, US

(21) Appl. No.: 09/915,515

(22) Filed: Jul. 27, 2001

Related U.S. Application Data
(60) Provisional application No. 60/221,199, filed on Jul. 27, 2000.

(51) Int. Cl. C12Q 1/70; G06F 19/00; G01N 33/48; G01N 33/50

(52) U.S. Cl. 435/5; 702/19

(57) ABSTRACT

Novel paramyxovirus hemagglutinin-neuraminidase active site and methods for enabling the design and selection of inhibitors with that active site are provided. Also provided is machine-readable data storage medium comprising structure coordinates of the novel paramyxovirus hemagglutinin-neuraminidase active site.

### DESCRIPTION

1. Technical Field

The structures of Hemagglutinin-Neuraminidase and a Hemagglutinin-Neuraminidase: DANA complex from Newcastle Disease Virus, a member of the Paramyxovirus family, have been determined by x-ray crystallography. The invention relates to the identification of a novel paramyxovirus hemagglutinin-neuraminidase active site and methods for enabling the design and selection of inhibitors with that active site. The present invention also relates to machine-readable data storage medium comprising structure coordinates of the novel paramyxovirus hemagglutinin-neuraminidase active site.

2. Background of Invention

Viruses of the family Paramyxoviridae are enveloped negative-stranded RNA viruses which comprise two subfamilies, Paramyxovirinae and Pneumovirinae. The subfamily Paramyxovirinae includes Human Parainfluenza Viruses types 1, 2, 3, and 4, Mumps Virus, Newcastle Disease Virus, and Measles Virus. Human Respiratory Syncytial virus is a member of the subfamily Pneumovirinae.

As a group, these viruses are a leading cause of respiratory disease in humans, especially children. These paramyxoviruses are the causative agents of such respiratory diseases as croup, bronchitis, and pneumonia. Taken together the various stains of paramyxoviruses are responsible for annual epidemics in humans. Re-infection in following years is common but less severe (Kass, ed., Studies in Infectious Diseases Research, The University of Chicago Press, Chicago (1975), pp. 51-64). Although some immunity develops through infection by the various strains of paramyxoviruses it is not sufficient to provide complete protection. The vaccines that have been developed have been shown to have only limited efficacy in the short term so that long term usage is completely ineffective (Choppin and Scheid, Rev. Infect. Dis 2:40-61 (1980); Norrby et al., J. Infect Dis. 132:262-269 (1975). The lack of an effective vaccine together with the epidemiological evidence of the occurrence of annual epidemics of paramyxovirus infection indicates the urgent need for the development of therapeutic agents.

All members of the Respirovirus genus of the Paramyxoviridae family have two surface glycoproteins: hemagglutininneuraminidase (HN) protein and Fusion (F) protein. The HN protein provides three main functions which include recognizing sialic acid containing receptors on cell surfaces, promoting fusion activity of the F protein which allows entry into the cell, and acting as a neuraminidase, removing sialic acid from progeny virus particles to prevent viral self-agglutination, thereby facilitating spread of the virus. This is in contrast to influenza A and B viruses which have sialic acid binding and fusion functions combined in the tetrameric hemagglutinin (HA) glycoprotein while the sialic acid hydrolysis functionality resides in the tetrameric neuraminidase (NA). The key role of the surface glycoproteins in the infectivity of the influenza virus, have led to the development of therapeutic agents directed against both HA and HN (Watowich, S. J.

Skehel, J. J., & Wiley, D. C Structure 2:719-731 (1994); von Itzstein, M. et al. Nature 363:418-423 (1993); Kim, C. U. et al. Journal of the American Chemical Society 119:681-690 (1997)). The similar role of the multifunctional HN makes it an attractive target for development of therapeutic agents against paramyoxviruses.

### SUMMARY OF INVENTION

The present invention relates to structures of paramyxovirus hemagglutinin-neuraminidases, as determined by x-ray crystallography, the use of such structures to solve the structure of paramyxovirus hemagglutinin-neuraminidase homologues, mutants, co-complexes, and other crystal forms and the use of such structures, their homologues, mutants, co-complexes, and other crystal forms, to design inhibitors of paramyoxvirus hemagglutinin-neuraminidases.

In one aspect, the present invention provides the novel structure and atomic coordinates of a paramyxovirus hemagglutinin-neuraminidase from Newcastle Disease Virus (Kansas strain] as determined by x-ray crystallography. Another aspect of the present invention is to use the atomic coordinates to reveal a unique active site which is distinct from other known sialidases or neuraminidases. A further aspect of the present invention is to show that this active site is representative of other paramyxovirus hemagglutinin-neuraminidases based on sequence homology of key active site amino acids.

An additional aspect of the present invention provides the structure and atomic coordinates of a paramyxovirus hemagglutinin-neuraminidase from Newcastle Disease Virus [Kansas strain] cocomplex with an inhibitor, 2-deoxy-2,3-dehydro-N-acetylneuraminic acid (DANA), as determined by x-ray crystallography. A further aspect of the present invention is to use the atomic coordinates of the co-complex to reveal the active site. Still another aspect of the present invention is to use the atomic coordinates of the co-complex to identify amino acids of the active site that interact with the bound inhibitor.

A further aspect of the present invention provides a comparison of the structures of an unbound paramyxovirus hemagglutinin-neuraminidase and a co-complex of paramyxovirus hemagglutininneuraminidase: DANA, as determined by x-ray crystallography. An additional aspect provides the identification of important amino acids in the active site in the comparison of the structures. Yet another aspect of the present invention provides for the use of the structure of a paramyxovirus hemagglutinin-neuraminidase and/or a paramyxovirus hemagglutininneuraminidase co-complex to solve the structure of a homologue, mutant, co-complex, or other crystal form of a paramyxovirus hemagglutinin-neuraminidase.

A further aspect of the present invention provides a method to use the structure and atomic coordinates of a paramyxovirus hemagglutinin-neuraminidase, homologue, mutant, co-complex, or other crystal form, to design, evaluate computationally, synthesize and use inhibitors of hemagglutinin-neuraminidases that prevent or treat the undesired properties of infection by paramyoxviruses. A still further aspect of the present invention provides the use of these inhibitors of paramyxovirus hemagglutinin-neuraminidases as therapies that are beneficial in the treatment, or prevention of, croup, bronchitis, pneumonia, or any other respiratory disease caused by paramyxoviruses.

The present invention also relates to machine-readable data storage medium comprising structure coordinates of the novel paramyxovirus hemagglutinin neuraminidase active site.

Still other objects and advantages of the present invention will become readily apparent by those skilled in the art from the following detailed description, wherein it is shown and described preferred embodiments of the invention, simply by way of illustration of the best mode contemplated of carrying out the invention. As will be realized the invention is capable of other and different embodiments, and its several details are capable of modifications in various obvious respects, without departing from the invention. Accordingly, the description is to be regarded as illustrative in nature and not as restrictive.

### BRIEF DESCRIPTION OF DRAWINGS

FIG. 1 represents a ribbon diagram of the Newcastle Disease Virus Hemagglutinin-Neuraminidase: DANA co-complex. DANA, represented by a space-filling drawing, is in the active site in the center of the figure.

FIG. 2 is the secondary structure assignment and alignment of HN sequences from 7 representative paramyxoviruses: Sendai Harris strain (sendai), Human parainfluenza virus type 1 strain Washington/1957 (PIV1), Human parainfluenza virus type 3 strain Aus/124854/74 (PIV3), Simian Virus strain W3 (SV5), Human parainfluenza type 2 (PIV2), Mumps virus strain Miyahars vaccine (Mumps), and Newcastle disease virus strain Kansas (NDV). Residues conserved across all strains are shaded. The connectivity of the disulphide linkages is indicated by the single digit numbers above the Sendai sequence. The location of beta strands and alpha helices are shown below the NDV sequence. The nomenclature of BiSj indicates the j'th strand in the I'th beta-sheet The lines under the NDV sequence show residues involved in formation of the dimer interface: the upper line refers to the hexagonal crystal form, the lower line refers to the orthorhombic crystal form.

FIG. 3 shows key amino acids of the active site of the NDV HN:DANA co-complex. DANA is shown as a ball-and-stick drawing while the side chains of key amino acids are drawn with sticks.

FIG. 4 shows the active sites of both the orthorhombic pH 4.6 crystal structure and the hexagonal pH 6.1 HN:DANA co-complex crystal structure superimposed. The hexagonal form is drawn in dark gray and the orthorhombic form is drawn in light gray. The main conformational changes involve R174, D198, and K236.

FIG. 5—Crystallographic Data Collection Statistics

### ABBREVIATIONS AND DEFINITIONS

In order to facilitate an understanding of the present invention, the following listings of Abbreviations and Definitions are provided:

### Abbreviations

NDV=Newcastle Disease Virus

HN=Hemagglutinin-Neuraminidase

cHN=Globular head of hemagglutinin-neuraminidase proteolyticaly cleaved from membrane bound hemagglutinin-neuraminidase.

NA=Neuraminidase

HA=Hernagglutinin

DANA=Neu5Ac2en=2-deoxy-2,3-dehydro-N-acetyineuraminic acid

Amino Acids

A=Ala=Alanine

V=Val=Valine

L=Leu=Leucine

I=Ile=lsoleucine

P=Pro=Proline

F=Phe=Phenylalanine

W=Trp=Tryptophan

M=Met=Methionine

G=Gly=Glycine

S=Ser=Serine

T=Thr=Threonine

C=Cys=Cysteine

Y=Tyr=Tyrosine

N=Asn=Asparagine

Q=Gln=Glutamine

D=Asp=Aspartic Acid

E=Glu=Glutamic Acid

K=Lys=Lysine

R=Arg=Arginine

H=His=Histidine

### Definitions

The term “homologue” means a protein having at least 30% amino acid sequence identity with a paramyxovirus hemagglutinin-neuraminidase or any functional domain of a paramyxovirus hemagglutinin-neuraminidase.

The term “mutant” refers to a paramyxovirus hemagglutinin-neuraminidase polypeptide, i.e., a polypeptide displaying the biological activity of wild-type paramyxovirus hemagglutininneuramindase , characterized by the replacement of at least one amino acid from the wild-type HN sequence. Such a mutant may be prepared, for example, by expression of HN cDNA previously altered in its coding sequence by oligonucleotide-directed mutagenesis. Mutants may also be generated by site-specific incorporation of unnatural amino acids into HN proteins using the general biosynthetic method of C. J. Noren et al., Science 244:182-188 (1989). In this method, the codon encoding the amino acid of interest in the wild-type paramyxovirus is replaced by a “blank” nonsense codon, TAG, using oligonucleotide-directed mutagenesis. A suppressor tRNA directed against this codon is then chemically aminoacylated in vitro with the desired unnatural amino acid. The aminoacylated tRNA is then added to an in vitro translation system to yield a mutant HN with the site-specific incorporated unnatural amino acid. In addition, selenocysteine or selenomethionine may be incorporated into wild-type or mutant HN by expression of HN cDNAs in auxotrophic E coli strains [W. A. Hendrickson et al. EMBO J 9(5):1665-1672 (1990)]. In this method, the wild-type or mutagenized FIN cDNA may be expressed in a host organism on a growth medium depleted of either natural cysteine or methionine (or both) but enriched in selenocysteine or selenomethionine (or both)

The term “active site” or “active site moiety” refers to any or all of the following sites in HN: the substrate binding site, the site where DANA binds, or the site where hydrolysis of a substrate occurs. The active site is characterized by at least amino acid residues 174, 175, 190, 192, 199, 234, 236, 237, 254, 256, 258, 262, 299, 302, 317, 363, 364, 369, 401,416, 466, 498, and 526 using the sequence and numbering according to Takimoto,T., Taylor,G. L., Crennell,S. J., Scroggs,R. A. and Portner,A. (submitted, SEQ ID NO:1) The term “co-complex” means a paramyxovirus hemagglutinin-neuraminidase or a mutant or homologue of a paramyxovirus hemagglutinin-neuraminidase in covalent or non-covalent association with a chemical entity or compound,

The term “heavy atom derivative” refers to derivatives of paramyxovirus hemagglutinin-neuraminidase produced by chemically modifying a crystal of HN. In practice, a crystal is soaked in a solution containing heavy metal atom salts, or organometallic compounds, e.g., lead chloride, gold thiornalate, thimerosal or uranyl acetate, which can diffuse through the crystal and bind to the surface of the protein. The location(s) of the bound heavy metal atom(s) can be determined by x-ray diffraction analysis of the soaked crystal. This information, in turn, is used to generate the phase information used to construct the three-dimensional structure of the protein.

The term “structure coordinates” refers to mathematical coordinates derived from mathematical equations related to patterns obtained on diffraction of a monochromatic beam of X-rays by the atoms of an HN molecule in a crystal form. An electron density map of the repeating unit of the crystal is calculated from the diffraction data. The positions of each atom can be established from the electron density map.

The term “associating with” indicates the proximity between a chemical entity or compound, or portions thereof, and a HN molecule or portions thereof. The association may be non-covalent such that the positions are energetically favorable or it may be covalent.

The “kinetic form” of HN refers to the condition of the enzyme in its free or unbound state or bound to a chemical entity at either its active site or accessory binding site.

A “competitive” inhibitor refers to an inhibitor that inhibits HN activity by binding to to the same kinetic form of HN as its substrate. Such an inhibitor directly competes with the substrate for the active site of HN. Increasing the substrate concentration has the effect of reversing the inhibition of a competitive inhibitor.

An “uncompetitive” inhibitor binds to a different kinetic form of HN than does the substrate to give inhibition. These types of inhibitors bind to HN which already has a substrate molecule bound and do not bind to free HN. Changing the substrate concentration has no effect on the inhibition of an uncompetitive inhibitor.

A “non-competitive” inhibitor can bind to either free or substrate bound HN The identification of inhibitors as competitive, uncompetitive, or non-competitive is possible by those of skill in the art by computer fitting enzyme kinetic data using standard equations according to Segel, I. H., Enzyme Kinetics, J. Wiley & Sons, (1975).

### BEST AND VARIOUS MODES FOR CARRYING OUT INVENTION

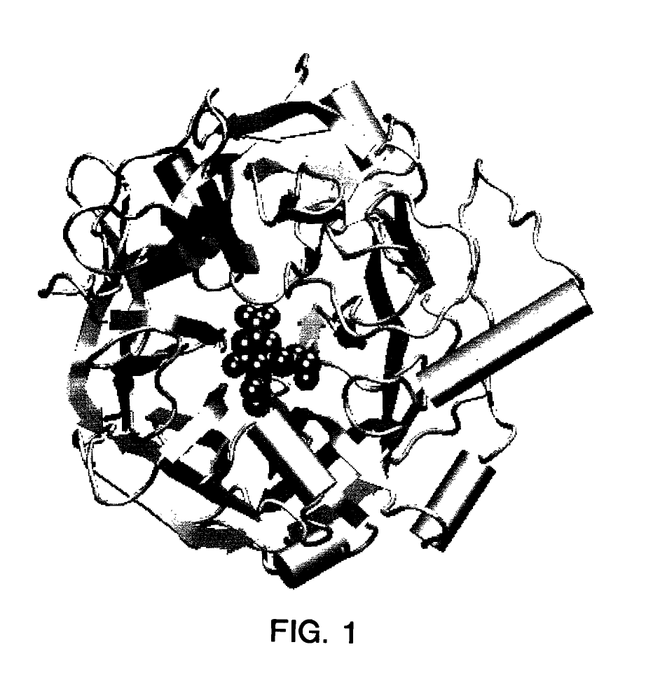

In order to facilitate the development of therapeutic agents for the treatment or prophylaxis of paramyxovirus infection, the crystal structure of Hemagglutinin-Neuramindase from Newcastle Disease Virus [Kansas Strain] (NDV) was determined. The crystallization of the HN has been previously disclosed in WO 97/09345. The structure of the HN is a six bladed B-propeller (FIG. 1), a topology shared with other HN, NA, and bacterial neuraminidases (Taylor, G. Current Opinion in Structural Biology 6:830-837 (1996)). To further aid in the discovery of therapeutic agents, the co-complex crystal structure of Hemagglutin in-Neuraminidase: DANA was also determined by Xray crystallography.

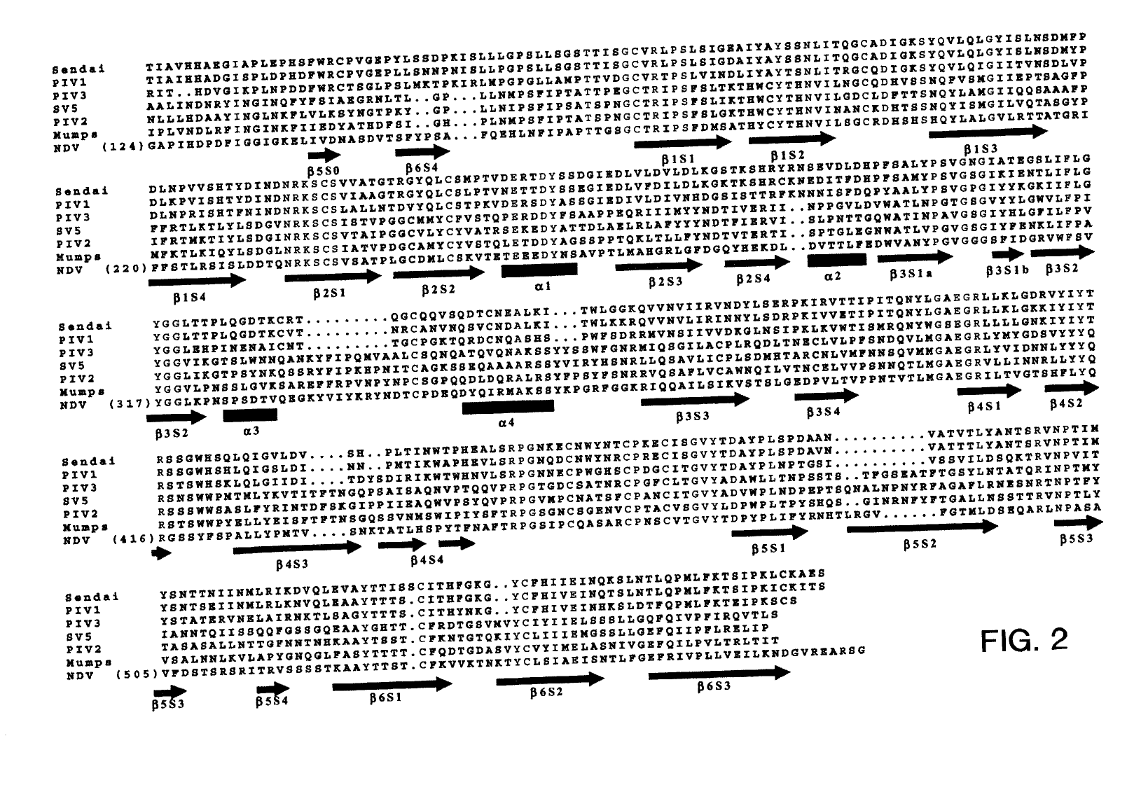

Within the Paramyxoviridae family HN sequence identities range from 25% to 75% between species. Therefore, the structure of HN from NDV disclosed is representative of HN from all paramyoxviruses. This assertion is supported by the conservation of amino acids located in and near the active site for all members of the paramyoxvirus family (see FIG. 2). The unexpected novelty of this prototypic paramyoxvirus HN active site determined from NDV is the presence of several distinctive structural features that have not been observed in other viral or bacterial sialidases. These features suggest that therapeutic agents for paramyoxvirus HN would have to be chemically distinct from current therapeutic agents such as those directed against influenza neuraminidase. In support of this assertion, example compounds are disclosed which have excellent biological activity toward influenza neuraminidases but little or no biological activity on paramyoxvirus hemagglutinin-neuramindases.

The following non-limiting examples are presented to further illustrate the present invention.

### EXAMPLES

### Example 1

### Isolation and Purification

Newcastle Disease Virus (strain Kansas) is grown in embryonated eggs. The membrane proteins of the virus are isolated by disruption with Triton X-100 and reconstituting into virosomes, After sedimentation of the ribonucleaprotein complex, the supernatant containing viral membrane proteins with lipids and Triton X-100 is isolated. Removal of the detergent using Bio-Beads reconstituted the virosomes containing the membrane proteins. Virosomes are then treated with chymotrypsin at 500ug/mL to cleave hem agglutin in-neuram inidase from the virosome. A solution containing the cleaved HN is purified by filtration through Centricon 100 yielding pure concentrated cHN. Analysis by SDS-PAGE under non-reducing conditions shows that cHN migrates as a monomer, suggesting that there are no disulfide linkages between monomers.

### Example 2

### Crystallization

Crystallization of the cHN is carried out using vapor diffusion in hanging drops. A number of different crystal forms, including bipyramidal and rhomboid, were obtained in 10 a precipitant that contained PEG3350 and 0.2M ammonium sulphate in 0.1 M citrate buffer (pH 4.6).

Crystals of the cHN:DANA co-complex are obtained by incubating cHN (12 mg/mL) with sialic acid at the final concentration of 10 mM. The co-complex crystallizes in a precipitant that containes 20% PEG3350 in 0.1 hepes buffer (pH 6.2 or 6.4). The crystals grow as hexagonal pyramids or a combination of hexagonal prisms and pyramids.

Further details of the crystallization will be apparent to those skilled in the art and need not be discussed herein. In particular, see the crystallization conditions previously disclosed in WO 97/09345.

### Example 3

### Data Collection, Phasing, Model Building and Refinement

Crystals of cHN belong to the orthorhombic space group P2₁2₁2₁. The unit cell suggested two cHN molecules per asymmetric unit. Brief immersion of the crystals in the crystallization buffer with 10% v/v glycerol added allowed collection of data at 1 OOK. A search of potential heavy atom derivatives was hampered by the extreme non-isomorphism observed, with unit cell dimensions varying over the following limits:

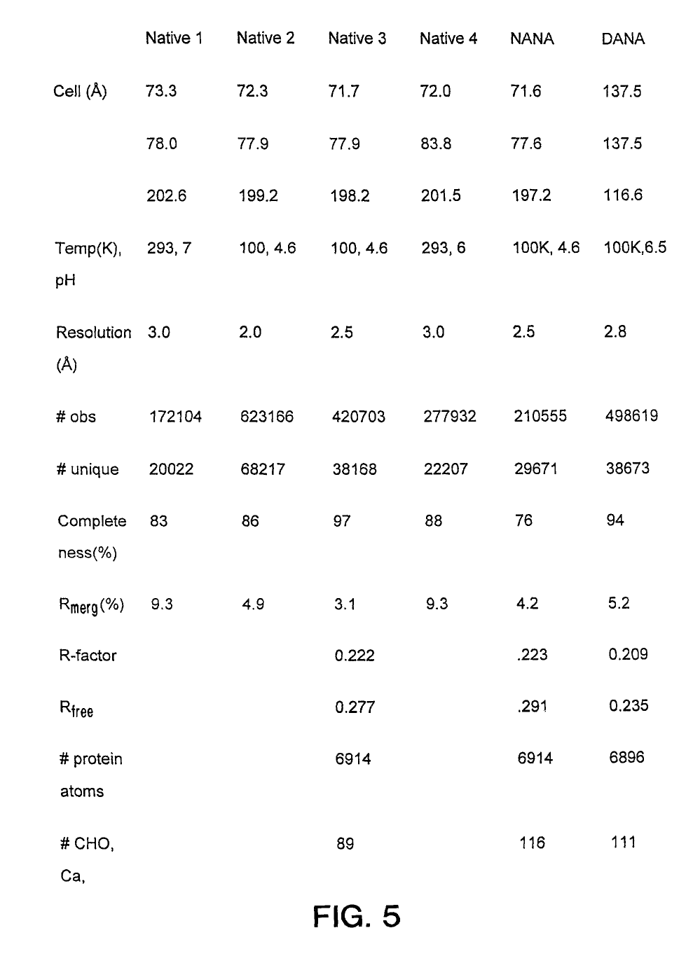

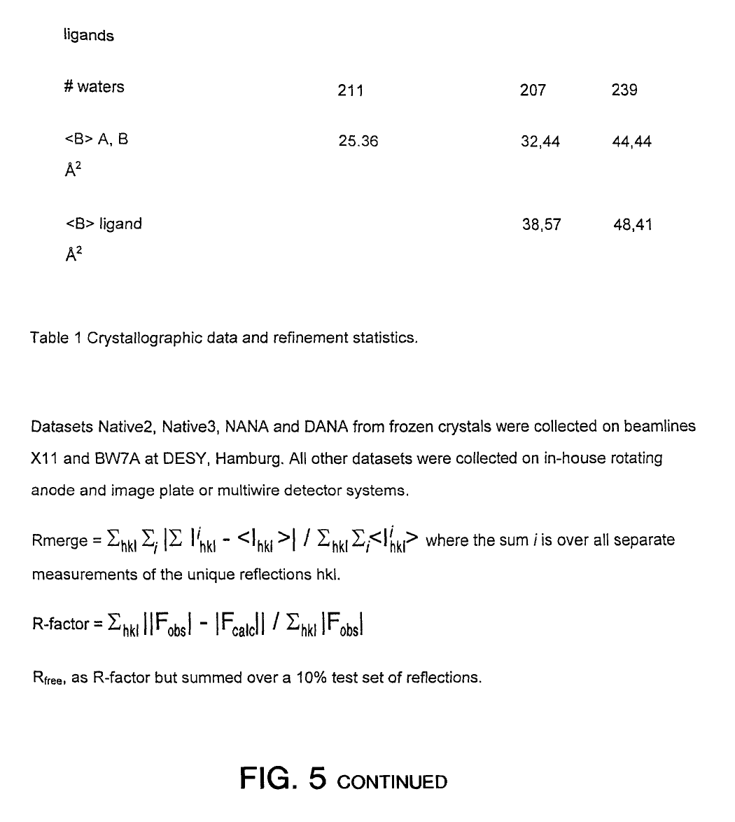

a=70.7-75.5A, b=71.8-87.o A, and c=l 94.6-205.5A. Progress in phasing was also hampered by the inability to find reliable freezing conditions for the crystals at higher pH which resulted in incompleteness of data. A low resolution phase set was assembled from eight heavy atom derivatives taken at room temperature that gave a mean figure of merit of 0.53 to 4A. The solvent-flattened electron density map revealed density consistent with the two cHN molecules in the asymmetric unit. However, attempts to phase extent to 10A using the room temperature native data, NATIVE1 (FIG. 5), failed to produce an interpretable map. In addition, the non-crystallographic symmetry relating the two cHN molecules could not be determined from self rotation functions or from native Patterson maps. The structure was finally determined by using the non-isomorphism to advantage. Four data sets were chosen that were to as high a resolution as possible, were as complete as possible, and were as non-isomorphous from one another as possible (>50% merging R-factors). DMMULTI (CCP4 suite, Acta Crystallographica D50:7607673 (1994)) was then used to cross-crystal average between the four crystals and to phase extend from 6.OA to the limits of the individual datasets; using crude masks for each cHN molecule derived by placing an influenza neuraminidase alpha-carbon model roughly into the 4.OA MIR map. The resulting electron density was spectacular and allowed rapid tracing of a partial alpha-carbon model for each monomer. These partial models were then used to determine the non-crystallographic symmetry relating the monomers, which was then used in another round of DMMULTI. The non-crystallographic symmetry is a 2-fold rotation axis with spherical polar angles (0=790, (P=860, resulting in it being hidden under one of the crystallographic screw axes in the self rotation function.

Interpretation of the structure was carried out using cycles of model building using 0 (Jones, A. T., Zhou, J. Y., Cowan, S. W. & Kjeldgaard, M. Acta Crystallographica A47:110-119 (1991)) and refinement using CNS (Brunger, A. T. et.al., Acta Crystallographica D54:905-921 (1998)). Initially this involved the highest resolution data, NATIVE2, however the refinement did not progress well, which was thought to be the result of the data being a merger of data from three crystals. All subsequent refinement was carried out using NATIVE3 to 2.5A, which was more complete and derived from a single crystal. The final model contains residues 124 to 569 in monomer A residues 124 to 572 in monomer B, one N-linked N-acetylgalactosamie (NAG) at residue 341 in monomer A, two N-linked NAGs at residue 481, a divalent metal (assumed calcium) in monomer A, three glycerol molecules and 211 water molecules. Monomer A and B have mean isotropic B. factors of 25A2 and 36A 2 respectively, indicating the increased relative flexibility of monomer B In the crystal that leads to the extreme non-isomorphism

The Neu5Ac co-complex (dataset NANA) crystals belong to the hexagonal space group P61 with two monomers per asymmetric unit. The crystals were cryoprotected by the addition of 20% glycerol to the crystallization buffer, allowing data collection at 1 OOK (dataset DANA). Molecular replacement using the refined orthorhombic structure was straightforward using AmoRE (CCP4 suite, Acta Crystallographica D50:760-763 (1994)). Refinement was again carried out using CNS with non-crystallographic symmetry restraints being maintained. The final model contains residue 124 to 570 in monomer A, residues 124 to 569 in monomer B, N-linked NAGs ar residues 341 and 481 in both monomers, one divalent metal ion per monomer (assumed calcium), one inhibitor (DANA) per monomer and 239 water molecules. The monomers have a mean isotropic B-factor of 44 A2.

All data were processed with HKL package (Otwinowski, Z. & Minor, W., Methods EnzymoL 276:307-326 (1996)) and all crystallographic calculations, apart from refinement, were made using the CCP4 program suite (CCP4 suite, Acta Crystallographica D50:760-763 (1994)). The x-ray coordinates of the native structure and complex structure Newcastle Disease Virus Hemagglutinin-Neuraminidase will be deposited with the Protein Data Bank.

### Example 5

### Structural Features

The structure of the monomer, shown in FIG. 1, reveals the canonical B-propeller fold seen in other neuraminidases/sialidases. FIG. 2 shows the location of secondary structural elements in the sequence, and the connectivity of the six disulfide bonds, five of which are conserved across all HNs. N-Linked glycosylation is observed in the crystal at residues 341 and 481, consistent with earlier observations (McGinnes, L. W. & Morrison, T. G., Virology 212:398-410 (1995)). A metal ion was identified in the electron density, most probably calcium, coordinated to main chain carbonyl oxygen atoms and the side chains of residues 261 and 264, residues that are largely conserved across all HNs. The ion appears to play a structural role, stabilizing the position of conserved residues involved in sialic acid recognition.

### Example 6

### Active Site

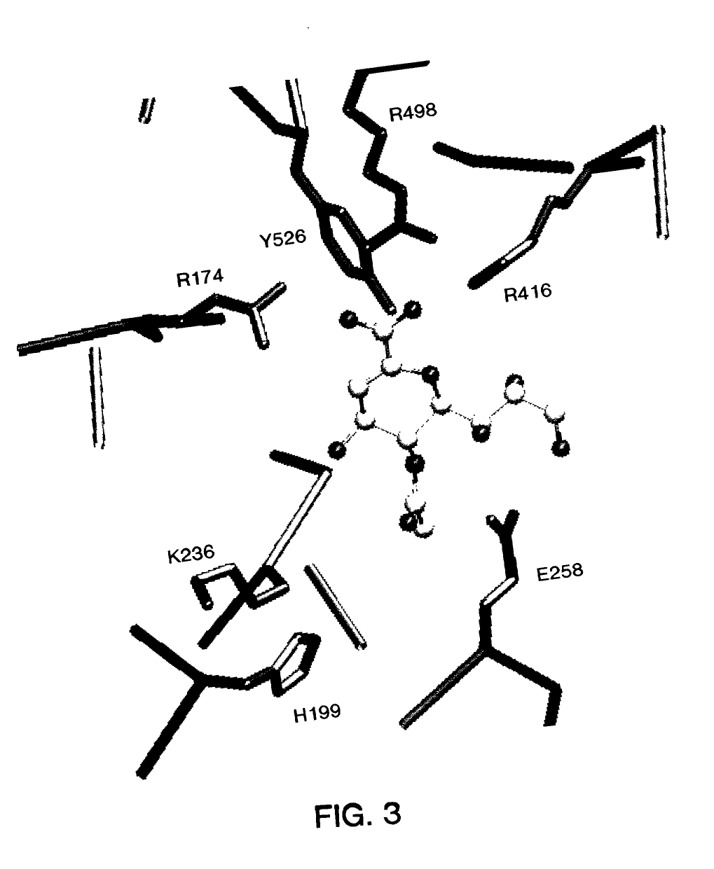

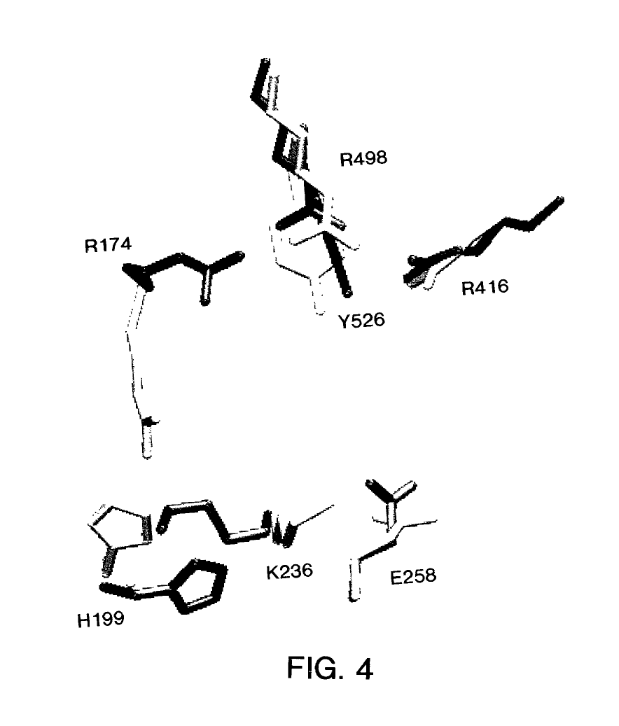

The most striking finding is that a single binding site on HN provides both the sialic acid binding and hydrolysis functions, and that the functionality appears to be controlled by a conformational switch. There is no evidence for a second sialic acid binding site, as suggested by much of the literature on HN, nor for a site equivalent to the hemagglutinin site found on the neuraminidase of some avian influenza virus strains (Varghese, J. N., PNAS 94:11808-11812 (1997)). The sialic acid co-complex shows the sugar bound in the chair conformation (FIG. 3), as observed in influenza hernagglutinin substrate complexes, rather than the strained boat conformation observed for sialic acid bound in the influenza neuraminidase. The residues interacting with the inhibitor are largely conserved across all HNs of the paramyxoviruses and are shown in FIG. 2, and suggest that the active site of this Newcastle Disease Virus HN serves as a prototypical model of paramyxoviruses HNs. Although the overall gross structure bears many similarities with other sialidase/neuraminidases the active site reveals the unexpected finding of novel interactions with the substrate that have never before been observed. This suggested that for paramyxovirus HNs the active site utilizes different modes of interaction with substrate in comparison with other neuraminidases such as influenza neuraminidase. Of particular note, in the DANA co-complex are the intereactions with all three hydroxyl groups of the glycerol chain, a feature never observed in other viral or bacterial sailidases so far studied. Also of note are the lack of intereactions with 04, N9, and 010 of the inhibitor. A structural comparison of the substrate and inhibitor complexes reveals major changes in the positions of conserved residues around the binding site and suggest a conformational switch that changes the role of the binding site (FIG. 4)

The active sites of all sialidase/neuraminidases whose structures have been determined possess several common features (Taylor, G., Current Opinion in Structural Biology 6:830-837 (1996)) and suggest a rigid pocket that induces strain in the conformation of the sialic acid sugar ring prior to hydrolysis (Taylor, N. R. & Vonitzstein, M., J. Med. Chem. 37:616-624 (1994)). In particular, the sialic acid carboxyl group interacts with three conserved arginines, one of which is held in place by a conserved glutamic acid, and the HN-inhibitor complex conforms to this pattern. In the HN-sialic acid co-complex, however, one member of the arginine triad, R174, is not in its “normal” position interacting with the glutamic acid, E547, but has swung around by 900 (FIG. 4). This substrate co-complex was obtained at pH 4.6, but it is believed that this new position is not an artifact of the low pH. In support of this, diffraction data was obtained from crystals grown at pH 4.6 but transferred to buffers at pH 6 and 7, where R174 remains in position. In addition, in this unusual position R174 participates in a hydrogen bonding network involving residues conserved across all HNs: R174→N19O→S202 and Q204→T188→H20→carbonyl oxygens of C238 and 1175. To allow R174 to make this dramatic movement, the active site of HN has an unusually large pocket, in comparison with the other sialidases, around the 04 position. Another key residue is the conserved lysine, K236, that also makes a dramatic movement: in the sialic acid complex it interacts with 010 of sialic acid and in the inhibitor complex (DANA) it moves into the environment previously occupied by R174 and becomes involved in the hydrogen bond network N190->S202->N234 and K236. Other key residues such as Y526 and 1175 also move significantly within the pocket. Y526 is the counterpart of the tyrosine which in conjunction with the conserved glutamic acid E401 in HN is thought to stabilize the cationic transition state intermediate. This suggests that one pocket can provide both sialic acid binding and hydrolysis.

When R174 is in its alternative position with the concomitant movement of 1175 and Y526, the pocket becomes simply a sialic acid binding site. Support for the role of K236 in this switch comes from mutation studies on the conserved NRKSCS motif, that includes N234, K236, and C238, where mutation of either N234 or K236 effectively abolishes neuraminidase activity and significantly reduces sialic acid binding ability (Mirza, A. M., Deng, R. T. & lorio, R. M., Journal of Virology 68:5093-5099 (1994)).

### Example 7

### Biological Assays

Influenza Neuraminidase Assay A fluorimetric assay was used to measure influenza virus NA activity. The substrate (2′-(4methylumbelliferyl)-al pha-D-acetylneuraminic acid) is cleaved by NA to yield a fluorescent product that can be quantified. The assay mixture contained inhibitor at various concentrations and NA enzyme in 32.5 mM MES (2-(N morpholino) ethanesulfonic acid) buffer, 4 mM calcium chloride at pH=6.5 (total volume=80 pl). The reaction was started by the addition of 20 pl of the substrate to a final concentration of 75 pM. After incubation for 30-90 min at 370C., 150 pl of 0.2 M glycine—NaOH, pH=10.2 was added to 0.1 ml reaction mixture to terminate the reaction, A blank was run with the same substrate solution with no enzyme. Fluorescence was recorded using spectrafluor (excitation: 360 nm and emission: 450 nm) and readings from substrate blanks were subtracted from the sample readings. The IC50 was calculated by plotting percent inhibition of NA activity versus the inhibitor concentration, Paramyxovirus Hemagglutinin Neuraminidase Assay A fluorimetric assay was used to measure paramyxovirus HN activity. The substrate (2′-(4methyl u m bell iferyl)-al pha-D-acetyl neuram in ic acid) is cleaved by HN to yield a fluorescent product that can be quantified. The assay mixture contained inhibitor at various concentrations and HN enzyme in 32.5 mM MES (2-(N morpholino) ethanesulfonic acid) buffer, 4 mM calcium chloride at pH=6.5 (total volume=80 μl). The reaction was started by the addition of 20 pl of the substrate to a final concentration of 75 μM. After incubation for 30-90 min (different times for different viruses) at 37° C., 150 μl of 0.2 M glycine—NaOH, pH=10.2 was added to 0.1 ml reaction mixture to terminate the reaction. A blank was run with the same substrate solution with no enzyme. Fluorescence was recorded using spectrafluor (excitation: 360 nm and emission˜450 nm) and readings from substrate blanks were subtracted from the sample readings. The IC₅₀ was calculated by plotting percent inhibition of HN activity versus the inhibitor concentration,

### Example 8

### Comparison of Biological Activity

The unexpected novelty of the NDV active site is the dramatic difference between it and the influenza neuraminidase active site. These differences, which are disclosed in example 6, suggest that influenza neuraminidase inhibitors would be poor NDV inhibitors. This is illustrated by the biological activity of the three following compounds

Compound 1:

alpha-3-[(Acetylamino)(diethylaminocarbonyl)methyl]-beta-4-[(aminoiminomethyl) amino]cyclopentane-1-carboxylic acid

Compound 2:

(-)-t-e-[(1-Acetylamino)(2-ethyl)butyl]-c-4-(aminoiminomethyl)amino-t-2-hydroxycyclopentan-r-1-carboxylic acid

Compound 3:

(-)t-3-[(1-Acetylamino-2-ethyl)butyl]-c-4-(aminoiminomethyl)aminocyclopentan-r-1-carboxylic acid

These compounds have been previously disclosed in WO97/47194 and WO99/33781. Testing for biological activity as described in example 7 gave the following results:

| | Influenza | Sendai Virus |
|---|---|---|
| | Neuraminidase | Hemagglutinin-Neuraminidase |
| Compound 1 | <0.05 uM | >300 uM |
| Compound 2 | <0.05 uM | >300 uM |
| Compound 3 | <0.05 uM | >300 uM |

Comparison of the biological activity of each compound shows significantly higher influenza neuraminidase activity in contrast to the low Paramyxovirus HN activity. The novel structure of paramyxovirus HN active sites are so distinct from influenza neuraminidase active sites that inhibitors of influenza neuraminidase are ineffective as therapeutic agents against paramyxoviruses.

The foregoing description of the invention illustrates and describes the present invention. Additionally, the disclosure shows and describes only the preferred embodiments of the invention but, as mentioned above, it is to be understood that the invention is capable of use in various other combinations, modifications, and environments and is capable of changes or modifications within the scope of the inventive concept as expressed herein, commensurate with the above teachings and/or the skill or knowledge of the relevant art. The embodiments described hereinabove are further intended to explain best modes known of practicing the invention and to enable others skilled in the art to utilize the invention in such, or other, embodiments and with the various modifications required by the particular applications or uses of the invention. Accordingly, the description is not intended to limit the invention to the form disclosed herein. Also, it is intended that the appended claims be construed to include alternative embodiments.

What is claimed is:

1. A method for identifying a potential inhibitor for a paramyxovirus Hemagglutinin-neuraminidase, comprising the steps of:
a, using a three-dimensional structure of the paramyxovirus hemagglutinen-neuraminidase as defined by the structure coordinates comprising the amino acid residues 174, 175, 190, 192, 199, 234, 236, 237, 254, 256, 258, 262, 299, 302, 317, 363, 364, 369, 401, 416, 466, 498 and 526 according to SEQ ID NO: 1;
b. applying the three-dimensional structure to design or select the potential inhibitor;
c. obtaining the potential inhibitor;
d. associating the potential inhibitor with the enzyme in the presence of a substrate to determine the ability of the potential inhibitor to inhibit the enzyme.

2. The method according to claim 1, wherein the potential inhibitor is designed de novo.

3. The method according to claim 1, wherein the potential inhibitor is designed from a known inhibitor.

4. The method according to claim 1, wherein the step of employing the three-dimensional structure to design or select the compound comprises the steps of:
a. identifying chemical entities or fragments capable of associating with the enzyme; and
b. assembling the identified chemical entities or fragments into a single molecule to provide the structure of the potential inhibitor.

5. The method of according to claim 4, wherein the potential inhibitor is designed de novo.

6. The method according to claim 4, wherein the potential inhibitor is designed from a known inhibitor.

7. A complex of a paramyxovirus Hemagglutinin-neuraminidase and a substrate or inhibitor molecule.

8. The complex of claim 7 being obtained by diffusion or co-crystallization.

9. The method according to claim 1 wherein the potential inhibitor is a competitive, noncompetitive, or uncompetitive inhibitor of a paramyoxvirus Hemagglutinin-Neuraminidase.

10. The method according to claim 1 wherein the potential inhibitor is designed to form salt links with Arg416 and Arg498 of a paramyxovirus hemagglutininneuraminidase.

11. The method according to claim 1 wherein the potential inhibitor is designed to form hydrogen bonds or salt links with Glu258 of a paramyxovirus hemagglutinin-neuraminidase.

12. The method according to claim 1 wherein the potential inhibitor is designed to form hydrogen bonds or salt links with Lys 199 of a paramyxovirus hemagglutinin-neuraminidase.

13. The method of claim 1 which comprises using a three-dimensional structure of paramyxovirus hemagglutnin-neuraminidase.

14. The method of claim 13 wherein the paramyxovirus hemagglutnin-neuraminidase is Neucastle Disease Virus hemagglutnin neuraminidase.

15. A machine readable data storage medium comprising a data storage material encoded with machine-readable data which comprises the structure coordinates comprising the amino acid residues 174, 175, 190, 192, 199, 234, 236, 237, 254, 256, 258, 262, 299, 302, 317, 363, 364, 369, 401, 416, 466, 498 and 526 according to SEQ ID NO: 1.

16. The machine-readable data storage medium of claim 15 wherein the machine-readable data comprises the three-dimensional structure of paramyxovirus Hemagglutnin-neuraminidase.

17. The machine-readable data storage medium of claim 16 wherein the paramyxovirus Hemagglutnin-neuraminidase is Newcastle Disease Virus Hemagglutnin-Neuraminidase.

18. A computer for producing a three-dimensional representation of:
a. a molecule or molecular complex, wherein said molecule or molecular complex comprises a binding pocket defined by structure coordinates comprising the amino acid residues 174, 175, 190, 192, 199, 234, 236, 237, 254, 256, 258, 262, 299, 302, 317, 363, 364, 369, 401, 416, 466, 498 and 526 according to SEQ ID NO: 1; wherein the computer comprises:
(i) a machine-readable data storage medium comprising a data storage material encoded with machine-readable data, comprising the structure coordinates comprising the amino acid residues 174, 175, 190, 192, 199, 234, 236, 237, 254, 256, 258, 262, 299, 302, 317, 363, 364, 369, 401, 416, 466, 498 and 526 according to SEQ ID: 1;
(ii) a working memory for storing instructions for processing the machine-readable data;
(iii) a central-processing unit coupled to the working memory and to the machine-readable data storage medium for processing the machine readable data into the three-dimensional representation; and
(iv) a display coupled to the central-processing unit for displaying the three-dimensional representation.

19. The computer of claim 18 wherein the computer produces a three-dimensional representation of a paramyxovirus Hemagglutnin-neuraminidase; and
wherein the machine-readable data comprises the structure coordinates of paramyxovirus Hemagglutnin-neuraminidase.

20. The computer of claim 18 wherein the computer produces a three-dimensional representation of Newcastle Disease Virus Hemagglutnin Neuraminidase and wherein the machine readable data comprises the structure coordinates of Newcastle Disease Virus Hemagglutnin Neuraminidase.

21. The method of claim 1 which comprises:
employing computational means to perform a fitting operation between the potential inhibitor and the structure coordinates of the three-dimensional structure; and
analyzing the results of the fitting operation to quantify the association between the potential inhibitor and the structure coordinates.


SEQUENCE LISTING (1 sequence)

SEQ ID NO: 1 - protein, 577 aa, Newcastle Disease Virus (Kansas Strain)
MDRAVSQVALENDEREAKNTWRLIFRIAILLLTVVTLATSVASLVYSMGASTPSDLVGIPTRISRAEEKITSALGSNQDVVDRIYKQVALESPLALLNTETTIMNAITSLSYQINGAANNSGWGAPIHDPDFIGGIGKELIVDNASDVTSFYPSAFQEHLNFIPAPTTGSGCTRIPSFDMSATHYCYTHNVILSGCRDHSHSHQYLALGVLRTTATGRIFFSTLRSISLDDTQNRKSCSVSATPLGCDMLCSKVTETEEEDYNSAVPTLMAHGRLGFDGQYHEKDLDVTTLFEDWVANYPGVGGGSFIDGRVWFSVYGGLKPNSPSDTVQEGKYVIYKRYNDTCPDEQDYQIRMAKSSYKPGRFGGKRIQQAILSIKVSTSLGEDPVLTVPPNTVTLMGAEGRILTVGTSHFLYQRGSSYFSPALLYPMTVSNKTATLHSPYTFNAFTRPGSIPCQASARCPNSCVTGVYTDPYPLIFYRNHTLRGVFGTMLDSEQARLNPASAVFDSTSRSRITRVSSSSTKAAYTTSTCFKVVKTNKTYCLSIAEISNTLFGEFRIVPLLVEILKNDGVREARSG